(19) United States
(12) Patent Application Publication
(10) Pub. No.: US 2009/0306106 A1
(43) Pub. Date: Dec. 10, 2009

(54) FORMS OF CRYSTALLINE LAPATINIB AND PROCESSES FOR PREPARATION THEREOF

(76) Inventors: Leonid METSGER, Beer-Sheva (IL); Ariel MITTELMAN, Elad (IL); Slavik YURKOVSKI, Kiryat-Gat (IL)

Correspondence Address: KENYON & KENYON LLP, ONE BROADWAY, NEW YORK, NY, 10004, US

(21) Appl. No.: 12/437,518

(22) Filed: May 7, 2009

Related U.S. Application Data
(60) Provisional application No. 61/053,465, filed on May 15, 2008.
(60) Provisional application No. 61/054,935, filed on May 21, 2008.
(60) Provisional application No. 61/091,992, filed on Aug. 26, 2008.

(51) Int. Cl. A61K 31/517; C07D 405/04

(52) U.S. Cl. 514/266.24; 544/293

(57) ABSTRACT

The present invention provides new crystalline forms of lapatinib base, Form X and Form Y, and amorphous lapatinib base, pharmaceutical compositions comprising the new crystalline forms of lapatinib base, and/or the amorphous lapatinib base, and processes for their preparation.

### RELATED APPLICATIONS

This application claims the benefit under 35 U.S.C. § 119(e) of U.S. Provisional Application Nos. 61/053,465, filed May 15, 2008; 61/054,935, filed May 21, 2008; and 61/091,992, filed Aug. 26, 2008, each of which is incorporated herein by reference in its entirety.

### FIELD OF THE INVENTION

The invention encompasses new crystalline forms of lapatinib base, amorphous lapatinib base, and process for preparation thereof.

### BACKGROUND OF THE INVENTION

Lapatinib ditosylate, N-[3-chloro-4-[(3-fluorophenyl)methoxy]phenyl]-6-[5-[(2-methylsulfonylethylamino)methyl]-2-furyl]quinazolin-4-amine ditosylate, has the following chemical structure:

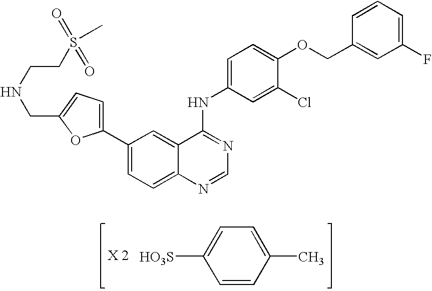

Lapatinib ditosylate is currently marketed in the United States under the tradename TYKERB® by GlaxoSmithKline. It was approved by the FDA as a drug for use in patients with advanced metastatic breast cancer.

Lapatinib ditosylate is described in PCT publications WO1999/035146, WO2002/002552, WO2005/046678, WO2006/113649, WO1998/002437, WO2001/004111, WO1996/009294, WO2002/056912, WO2005/105094, WO2005/120504, WO2005/120512, WO2006/026313, and WO2006/066267.

Lapatinib free base is described in U.S. Pat. No. 6,727,256.

The present invention relates to isolated solid lapatinib base. Free lapatinib base can be prepared and used as an intermediate for preparation of Lapatinib ditosylate. Additionally, free lapatinib base can be used in the processes for the preparation of other lapatinib salts.

The present invention relates to the solid state physical properties of lapatinib free base. These properties can be influenced by controlling the conditions under which lapatinib is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance. These conformational and orientation factors in turn result in particular intramolecular interactions such that different polymorphic forms may give rise to distinct spectroscopic properties that may be detectable by powder X-ray diffraction, solid state ¹³C NMR spectrometry and infrared spectrometry. A particular polymorphic form may also give rise to thermal behavior different from that of the amorphous material or another polymorphic form. Thermal behavior is measured in the laboratory by such techniques as capillary melting point, thermogravimetric analysis (TGA) and differential scanning calorimetry (DSC) and can be used to distinguish some polymorphic forms from others.

The discovery of new polymorphic forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic. There is a need in the art for additional polymorphic forms of lapatinib.

### SUMMARY OF THE INVENTION

The present invention encompasses solid lapatinib base. Preferably, the solid lapatinib base is a crystalline lapatinib base.

The present invention encompasses novel solid crystalline forms of lapatinib base referred to herein as Form X, and Form Y, and amorphous lapatinib base; processes for preparing thereof. The present invention also encompasses pharmaceutical compositions containing the solid lapatinib base, the crystalline lapatinib base, and/or one or more of lapatinib forms X, Y and amorphous lapatinib base.

### BRIEF DESCRIPTION OF THE FIGURES

FIG. 1 shows a powder X-ray diffraction pattern for Form X of lapatinib base.

FIG. 2 shows a powder X-ray diffraction pattern for Form Y of lapatinib base.

FIG. 3 shows a powder X-ray diffraction pattern for amorphous lapatinib base.

### DETAILED DESCRIPTION OF THE INVENTION

As used herein, the terms “slurry”, or “suspension” refer to a mixture of suspended solids in liquid (solvent). Typically, the solvent is used in an amount that does not result in the full dissolution of the substance.

As used herein, a “wet crystalline form” refers to a polymorph that was not dried using any conventional technique.

As used herein, a “dry crystalline form” refers to a polymorph that was dried using any conventional technique.

As used herein, drying is carried out at elevated temperature under reduced pressure. Preferably, the crystalline form is dried at about 30° C. to about 60° C., more preferably, between about 35° C. and about 55° C., and, most preferably, about 35° C. Preferably the drying is carried out under reduced pressure (for example less than 1 atmosphere, more preferably, about 10 mbar to about 100 mbar, more preferably, about 10 mbar to about 25 mbar). Preferably the drying takes place over a period of about 8 hours to about 50 hours, more preferably, about 10 hours to about 24 hours, and, most preferably, about 12 hours.

The present invention encompasses solid lapatinib base.

The present invention also encompasses crystalline lapatinib base.

In another embodiment, the invention encompasses crystalline Form X of lapatinib characterized by data selected from the group consisting of: a PXRD pattern with peaks at about 6.9, 11.4, and 16.0±0.2 degrees 2-theta, and at least two peaks at positions selected from the group consisting of 4.6, 20.0, 21.4, 22.9, 25.2, 27.5, and 32.2±0.2 degrees 2-theta; a PXRD pattern with peaks at about 20.0, 21.3, 24.0, 24.6 and 27.0±0.2 degrees 2-theta; and a PXRD pattern with peaks at about 6.8, 11.4, 16.0, 16.9, 18.0, 20.0, 21.3, 24.0, 24.6 and 27.0±0.2 degrees 2-theta.

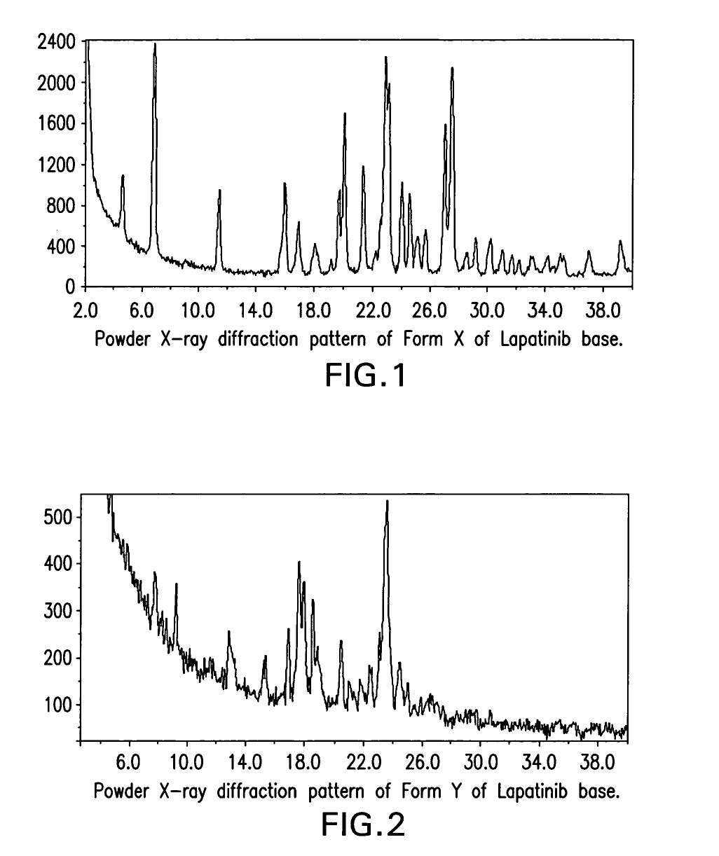

In one embodiment, the present invention encompasses crystalline Form X of lapatinib as characterized by the PXRD pattern illustrated in FIG. 1.

In another embodiment, the invention encompasses crystalline Form Y of lapatinib characterized by a PXRD pattern with peaks at about 7.8, 9.2, and 17.5±0.2 degrees 2-theta, and at least two peaks at positions selected from the group consisting of 12.8, 15.2, 16.8, 17.9, 18.5, 20.4, and 23.5±0.2 degrees 2-theta.

In another embodiment, the present invention encompasses crystalline Form Y of lapatinib as characterized by the PXRD pattern illustrated in FIG. 2.

In another embodiment, the invention encompasses a process for preparing Form X of lapatinib base comprising introducing lapatinib salts, preferably, lapatinib ditosylate into an organic solvent selected from the group consisting of acetonitrile, acetone, tetrahydrofuran, C1-C4 alcohols, and mixtures thereof, adding water; maintaining the resulting mixture for a sufficient period of time; and adding an inorganic base to obtain lapatinib base Form X.

Preferably, the mixture can be heated to about 30° C. to about 40° C. prior and/or after the addition of the base. Preferably, the base is an inorganic base selected from the group consisting of: alkali carbonates, alkali bicarbonates, and alkali hydroxides, most preferably the base is sodium carbonate. Lapatinib base Form X can be recovered by any conventional method, such as filtration.

Preferably, the obtained lapatinib base is dried.

In another embodiment, the invention encompasses a process for preparing Form Y of lapatinib base comprising introducing a composition of lapatinib base, into 1,4-dioxane; and heating the mixture at reflux for a sufficient period of time to obtain lapatinib base Form Y.

Lapatinib base Form Y can be recovered by any known method, such as filtration.

The present invention further encompasses amorphous lapatinib base.

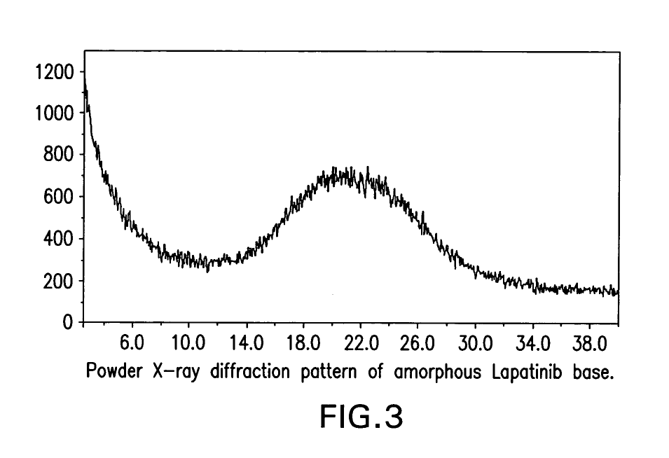

The amorphous lapatinib base is characterized by the PXRD pattern illustrated in FIG. 3.

In one embodiment, the present invention encompasses a process for preparing amorphous lapatinib base comprising providing a solution of lapatinib base and acetone; and removing the acetone by spray drying.

The term “spray drying” broadly refers to processes involving breaking up liquid mixtures into small droplets (atomization) and rapidly removing solvent from the mixture. In a typical spray drying apparatus, there is a strong driving force for evaporation of acetone from the droplets, which may be provided by providing a drying gas. Spray drying processes and equipment are described, for example, in Perry's Chemical Engineer's Handbook, pgs. 20-54 to 20-57 (Sixth Edition 1984).

By way of non-limiting example only, a typical spray drying apparatus comprises a drying chamber, atomizing means for atomizing a solvent-containing feed into the drying chamber, a source of drying gas that flows into the drying chamber to remove solvent from the atomized-solvent-containing feed, an outlet for the products of drying, and product collection means located downstream of the drying chamber. Examples of such apparatuses include Niro Models PSD-1, PSD-2 and PSD-4 (Niro A/S, Soeborg, Denmark). Typically, the product collection means includes a cyclone connected to the drying apparatus. In the cyclone, the particles produced during spray drying are separated from the drying gas and evaporated solvent, allowing the particles to be collected. A filter may also be used to separate and collect the particles produced by spray drying. Spray-drying may be performed in a conventional manner in the processes of the present invention (see, e.g., Remington: The Science and Practice of Pharmacy, 19th ed., vol. II, pg. 1627, herein incorporated by reference). The drying gas used in the invention may be any suitable gas, although inert gases such as nitrogen, nitrogen-enriched air, and argon are preferred. Nitrogen gas is a particularly preferred drying gas for use in the process of the invention. The lapatinib product produced by spray-drying may be recovered by techniques commonly used in the art, such as using a cyclone or a filter.

The lapatinib in the solution may be any crystalline or other forms of lapatinib, including various solvates and hydrates, as long as amorphous lapatinib base is produced during the spray drying process of the invention. When in solution, the crystalline form of the starting material does not affect the final result since the original form is lost.

Preferably, the lapatinib base used in the process is lapatinib base Form X. Preferably, the mixture is heated to induce dissolution; preferably the mixture is heated to about reflux.

The invention further provides a pharmaceutical formulation comprising the above described lapatinib base crystalline and/or amorphous forms. This pharmaceutical composition may additionally comprise at least one pharmaceutically acceptable excipient.

The invention further provides a pharmaceutical formulation comprising the above described lapatinib base crystalline and/or amorphous forms made by the processes of the present invention, and one or more pharmaceutically acceptable excipients. The compositions of the invention include powders, granulates, aggregates and other solid compositions comprising the form of lapatinib solid of the present invention.

The present invention also provides methods of treating metastatic breast cancer in a patient, preferably a human, by administrating to the patient a pharmaceutical composition comprising the lapatinib base crystalline form as described herein. Preferably, the pharmaceutical composition comprises a therapeutically effective amount of lapatinib base crystalline form.

The present invention also provides the use of the above described lapatinib base crystalline and/or amorphous forms for the manufacture of a pharmaceutical composition for the treatment of metastatic breast cancer.

The present invention also encompasses the use of the above described lapatinib base crystalline and/or amorphous forms for the preparation of lapatinib ditosylate.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### EXAMPLES

### X-Ray Power Diffraction

X-Ray powder diffraction data was obtained by using methods known in the art using a SCINTAG powder X-Ray diffractometer model X'TRA equipped with a solid-state detector. Copper radiation of 1.5418 Å was used. A round aluminum sample holder with zero background was used. The scanning parameters included: range: 2-40 degrees two-theta; scan mode: continuous scan; step size: 0.05 deg.; and a rate of 3 deg/min. All peak positions are within ±0.2 degrees two theta.

### Example 1

### Preparation of Lapatinib Base Form X

In a 500 ml round-bottomed flask equipped with a mechanical stirrer and a condenser were added 10.8 g of lapatinib ditosylate and 60 ml of acetonitrile. The resulting suspension was stirred at 40° C. for 1 hour. A solution of 1.24 gr of sodium carbonate in 70 ml of water was added drop-wise during 5 minutes. The resulting yellow suspension was stirred at 40° C. for 1 hour. Then, it was stirred at 25° C. for 2 hours, at 5° C. for 0.5 hour. The product was filtered in vacuum and dried for 48 hours in a vacuum oven at 35° C. Yield: 6.2 g (91%).

### Example 2

### Preparation of Lapatinib Base Form Y

To a 50 mg of solid lapatinib base Form X sample, 0.75 ml dioxane was added and a yellow solution was obtained during reflux. The resulting solution was cooled to 25° C., to obtain a yellow suspension. The resulting suspension was stirred over 1 hour at 25° C., whereupon it was filtered. The cake thus obtained was identified as Form Y of lapatinib base.

### Example 3

### Preparation of Amorphous Lapatinib Base

250 ml vessel was charged with 3 gr of lapatinib base Form X and 150 ml of acetone. It was heated to reflux and stirred until a clear solution was obtained. The solution was spray-dried in BUCHI instrument using the following parameters:

### Tᵢₙ—6° C.

### Tₒᵤₜ—6° C.

### Aspirator—100%

### Feed Rate—5%

Gas flow N₂—32 mm
Amorphous lapatinib base was obtained.

What is claimed is:

1. A solid lapatinib base.

2. A crystalline lapatinib base.

3. A crystalline form of lapatinib base (Form X) characterized by data selected from the group consisting of: a PXRD pattern having peaks at about 6.9, 11.4, and 16.0±0.2 degrees 2-theta, and at least two peaks at positions selected from the group consisting of 4.6, 20.0, 21.4, 22.9, 25.2, 27.5, and 32.2±0.2 degrees 2-theta; a PXRD pattern with peaks at about 20.0, 21.3, 24.0, 24.6 and 27.01±0.2 degrees 2-theta; and a PXRD pattern with peaks at about 6.8, 11.4, 16.0, 16.9, 18.0, 20.0, 21.3, 24.0, 24.6 and 27.0±0.2 degrees 2-theta.

4. The crystalline form of lapatinib base of claim 3 having an X-ray diffraction diagram substantially as depicted in FIG. 1.

5. A process for preparing the crystalline form of lapatinib base of claim 3, comprising introducing lapatinib salts into an organic solvent selected from the group consisting of acetonitrile, acetone, tetrahydrofuran, C₁-C₄ alcohols, and mixtures thereof; adding water; and adding an inorganic base to obtain lapatinib base Form X.

6. The process of claim 5, wherein the lapatinib salt is lapatinib ditosylate.

7. The process of claim 5, wherein the inorganic base is selected from the group consisting of alkali carbonate, alkali bicarbonate, and alkali hydroxide.

8. The process of claim 7, wherein the inorganic base is sodium carbonate.

9. The process of claim 5, further comprising drying the obtained lapatinib base.

10. The process of claim 9, wherein the drying the obtained lapatinib base is carried out at about 35° C. under reduced pressure.

11. A crystalline form of lapatinib base (Form Y) characterized by a PXRD pattern having peaks at about 7.8, 9.2, and 17.5±0.2 degrees 2-theta, and at least two peaks at positions selected from the group consisting of 12.8, 15.2, 16.8, 17.9, 18.5, 20.4, and 23.5±0.2 degrees 2-theta.

12. The crystalline form of lapatinib base of claim 11, having an X-ray diffraction diagram substantially as depicted in FIG. 2.

13. A process for preparing the crystalline form of lapatinib base of claim 11, comprising forming a solution of lapatinib base in dioxane; and heating the solution at reflux for a sufficient period of time to obtain the lapatinib base.

14. An amorphous lapatinib base.

15. A process for preparing amorphous lapatinib base, comprising providing a solution of lapatinib base in acetone; and removing the acetone by spray drying.

16. A pharmaceutical composition comprising the solid lapatinib base of claim 1, the crystalline lapatinib base of claim 2, the crystalline form of lapatinib base of claim 3 or claim 11, or the amorphous lapatinib base of claim 14.
